(19) United States
(12) Patent Application Publication
(10) Pub. No.: US 2017/0211037 A1
(43) Pub. Date: Jul. 27, 2017

(54) METHODS FOR PRODUCING BROWN ALGAE OF REDUCED IODINE CONTENT

(71) Applicant: MARUHA NICHIRO CORPORATION, Tokyo (JP)

(72) Inventors: Yosuke CHIBA, Ibaraki (JP); Hisashi YOSHIOKA, Ibaraki (JP); Takumi DAIMON, Saitama (JP); Hiroshi ODA, Ibaraki (JP); Masahiko SHIMADA, Tokyo (JP); Kuniaki TATSUTA, Tokyo (JP)

(73) Assignee: MARUHA NICHIRO CORPORATION, Tokyo (JP)

(21) Appl. No.: 15/515,029

(22) PCT Filed: Sep. 29, 2015
(86) PCT No.: PCT/JP2015/077428

§ 371 Date: Mar. 28, 2017

(30) Foreign Application Priority Data
Sep. 29, 2014 (JP) 2014-199232

(51) Int. Cl. C12N 1/12; A61K 36/02

(52) U.S. Cl. CPC C12N 1/12; A61K 36/02; C12N 2523/00; C12N 2527/00

(57) ABSTRACT

The present invention aims to provide methods for producing brown algae or brown algal extracts of reduced iodine content while retaining the structures of pigment components in the brown algae.

Provided is a method for producing a brown alga of reduced iodine content while retaining a pigment component in the brown alga, which method comprises shredding an untreated brown algal starting material and thereafter performing a heating treatment with hot water at 90-100° C. for 90-300 seconds. Also provided is a method for producing a brown alga of reduced iodine content while retaining a pigment component in the brown alga, wherein the brown alga obtained in the above-mentioned process is subjected to extraction using hydrous ethanol at a concentration of 60-100%.

### TECHNICAL FIELD

The present invention relates to methods for producing brown algae or brown algal extracts that have the iodine content reduced while retaining pigment components such as chlorophyll c and fucoxanthin in the brown algae; the present invention also relates to brown algae or brown algal extracts obtained by said methods.

### BACKGROUND ART

Brown algae contain pigment components such as chlorophyll c and fucoxanthin that are beneficiary to health and, if ingested continuously, they are expected to bring about various efficacies favorable to health. To be more specific, the Applicant recently found that chlorophyll c has a degranulation suppressing action and are useful in suppressing allergic diseases and bone joint diseases (see Patent Document 1). Heretofore, experiments have been conducted to administer chlorophyll c2 to animal models with allergic rhinitis, demonstrating its ability to alleviate allergic symptoms (Non-Patent Document 1). Fucoxanthin, on the other hand, has been reported to have an anti-obesity effect by assisting the burning of fat in fat tissues, as well as an anti-tumor action due to high apoptotic effect on cancer cells, a DHA synthesis promoting action, and anti-inflammatory actions (Non-Patent Documents 2-5).

Brown algae are also known to contain iodine in large amounts. Iodine is an essential element to humans since it is necessary for the synthesis of thyroid gland hormone which controls physiological processes such as reproduction, growth, and development. However, a continued excessive iodine intake will cause thyrotoxicosis and various other symptoms. Hence, an attempt to ingest brown algae continuously on a daily basis is hampered by the problem of excessive iodine intake. Therefore, a method is desired that is capable of reducing the iodine content of brown algae to a level just enough to meet human needs while maintaining the pigment components that are beneficiary to health.

According to the report of a study on the reduction of iodine content in brown algae, dried commercial products of kelp (Laminariaceae Bory), pinnatifida (Harvey) Suringar and fusiforme (Harvey) Setchell, in unshredded font', were immersed in deionized water for 20 and 60 minutes, giving the following data: based on the pre-immersion iodine content, the measured elutions were respectively 91.1±1.5 mg/100 g and 92.5±1.2 mg/100 g for kelp (Laminariaceae Bory), 22.6±9.8 mg/100 g and 27.7±7.7 mg/100 mg for pinnatifida (Harvey) Suringar, and 30.1±5.9 mg/100 g and 40.4±7.6 mg/100 g for fusiforme (Harvey) Setchell (see, for example, Non-Patent Document 6). It was also reported that when dry kelp (Laminariaceae Bory) was subjected to extraction in hot water at 80 ° C. for 5 minutes, iodine was extracted in more than 50% on average which varied with the part of kelp (see, for example, Non-Patent Document 7). However, each of these reports cites data from dried brown algae and to reduce the iodine content by the applicable methods, a raw brown alga as the starting material needs to be first dried with heat and then subjected to treatment with water, either at ordinary temperature or as heated, in order to remove iodine; this requires a multiple-stage process. The former approach has been shown to vary in effectiveness depending on the kind of seaweed and the iodine content may potentially be reduced only insufficiently. In the latter approach, more than one heating step is required, so heat-labile pigment components may potentially be decomposed.

### CITATION LIST

### Patent Literature

Patent Document 1: WO2013/100136A1

### NON-PATENT LITERATURE

Non-Patent Document 1: Yoshioka, H. et al., Inhibitory effect of chlorophyll c2 from brown algae saragassum horneri, on degranulation of RBL-2H3 cells, Journal of Functional Foods, 2013, 5: 204-210

Non-Patent Document 2: H. Maeda et al., Biochemical and Biophysical Research Communications, 332 (2005) 392-397

Non-Patent Document 3: Hosokawa, Bio Industry, 21 (2004) 52-57

Non-Patent Document 4: T. Tsukui et al., Journal of Agricultural and Food Chemistry, 55 (2007) 5025

Non-Patent Document 5: K. Shratori et al., Experimental Eye Research, 81 (2005) 422-428

Non-Patent Document 6: Nihon Eiyo Shokuryo Gakkai-shi (Journal of the Japan Society of Nutrition and Food Science), 36 (1), pp. 21-24, 1983

Non-Patent Document 7: Bulletin of Aoyama Gakuin Women's Junior College, 28, 121-125, 1974

### SUMMARY OF INVENTION

### Technical Problem

An object, therefore, of the present invention is to provide methods for producing brown algae or brown algal extracts of reduced iodine content while retaining the structures of pigment components in the brown algae, which methods are capable of efficiently extracting and recovering the pigment components from brown algae as the starting material and, at the same time, are also capable of conveniently reducing the content of iodine present in the brown algal starting material.

A further object of the present invention is to provide brown algae or brown algal extracts of reduced iodine content in the brown algae as obtained by the above-described production methods.

### Solution to Problem

With a view to attaining the above-stated objects, the present inventors made intensive studies using the brown alga hormeri (Turner) C. Agardh as a model and found that when a raw brown alga, namely, an untreated brown alga, was finely shredded and then heated by blanching for a brief period (90-300 seconds)—this is the first step—the iodine content could be reduced while pigment components retained their structures without being decomposed. From the brown alga as obtained in the first step, the pigment components were then extracted using aqueous solutions of ethanol at 60% and higher concentrations—this is the second step—the pigment components could be extracted with no iodine being transferred into the liquid extractant. The present invention was accomplished on the basis of these findings.

Briefly, the present invention lies in a method for producing a brown alga of reduced iodine content while retaining pigment components in the brown alga, which method comprises shredding an untreated brown algal starting material and thereafter performing a heating treatment with hot water at 90-100° C. for 90-300 seconds.

The present invention also lies in a method for producing a brown algal extract of reduced iodine content while retaining pigment components in the brown alga, which method comprises shredding an untreated brown algal starting material, thereafter performing a heating treatment with hot water at 90-100° C. for 90-300 seconds, and subjecting the resulting heat treated brown alga to extraction using hydrous ethanol at a concentration of 60-100%.

The present invention further lies in a brown alga or a brown algal extract as obtained by those production methods to have a reduced iodine content while retaining pigment components in the brown alga,

### Advantageous Effects of Invention

According to the methods of the present invention, there can be obtained brown algae or brown algal extracts that maintain pigment components and which yet have the iodine content reduced to levels that will not cause excessive iodine intake. Thus, the brown algae or brown algal extracts as produced by the methods of the present invention are so adapted that they contain pigment components beneficiary to health while at the same time they contain only ingestible amounts of iodine; as a result, they are suitable for use in such applications as foods intended for extended intake, in particular, functional foods.

### BRIEF DESCRIPTION OF DRAWINGS

FIG. 1 is a graph showing the percent residues of pigment components (chlorophyll c and fucoxanthin) and iodine in a brown alga as treated with hot water.

FIG. 2 is a graph showing in percentage how much of the pigment components (chlorophyll c and fucoxanthin) and iodine in a brown alga transferred into a liquid extractant that contained ethanol at varying concentrations and which was used to perform extraction after the treatment with hot water.

### DESCRIPOTION OF EMBODIMENTS

As noted above, the present invention is a method for producing a brown alga of reduced iodine content while retaining pigment components in the brown alga, which method comprises shredding an untreated brown algal starting material and thereafter performing a heating treatment with hot water at 90-100° C. for 90-300 seconds (the first step).

The brown algae that may be used as the starting material in the present invention are not particularly limited. Brown algae are seaweeds belonging to the Class Phaeophyceae in the Division Heterokontophyta and include the following seaweeds, namely brown algae of: the order ECTOCARPALES Setchell et Gardner, 1922, the family Ectocarpaceae C. Agardh, 1828, the genus Acinetospora Bornet, 1892 [Ectocarpus filamentosus, Ectocarpus ugoensis], the genus Asteronema Delepine et Asensi, 1975 nom. cons. [breviarticulatum (J. Agardh) Ouriques et Bouzon, Ectocarpus breviarticulatus, Feldmannia breviarticulata, Hincksia breviarticulata], the genus Ectocarpus Lyngbye, 1819 nom. cons. [arctus Kuetzing, confervoides, fusiformis Nagai, laurenciae Yamada, siliculosus (Dillwyn) Lyngbye, socialis Setchell et Gardner, yezoensis Yamada et Tanaka], the genus Feldmannia Hamel, 1939 [formosana (Yamada) Itono, Ectocarpus formosanus, irregularis (Kuetzing) Hamel, Ectocarpus irregularis, Ectocarpus izuensis], the genus Hincksia J. F. Gray, 1864 [granulosa (J. E. Smith) Silva, Ectocarpus granulosus, Giffordia granulosa, indica (Sonder) J. Tanaka, Ectocarpus indicus, Feldmannia indica, Giffordia indica, mitchellae (Harvey) Silva, Ectocarpus mitchellae, Giffordia mitchellae, Ectocarpus ovatus, Giffordia ovata, Ectocarpus sandrianus, Giffordia sandriana], the genus Laminariocolax Kylin, 1947 [aecidioides (Rosenvinge) Peters, Gononema aecidioides, Streblonema aecidioides], the genus Laminarionema Kawai et Tokuyama, the genus Spongonema Kuetzing, 1849 [tomentosum (Hudson) Kuetzing], the genus Streblonema Derbes et Solier in Castagne, 1851 [evagatum Setchell et Gardner, fasciculatum Thuret], the family Pylaiellaceae Pedersen, 1984, the genus Bachelotia (Bornet) Kuckuck ex Hamel, 1939 [antillarum (Grunow) Gerloff], the genus Pylaiella Bory, 1823 orth. cons. [littoralis (Linnaeus) Kjellman], the family Sorocarpaceae Pedersen, 1977, the genus Botrytella Bory, 1822 [parva (Takamatsu) Kim, micromora, Sorocarpus uvaefounis], the genus Polytretus Sauvageau, 1900 [reinboldii (Reinke) Sauvageau, Ectocarpus intricatus, Ectocarpus iwadatensis, Ectocarpus recurvatus]; the order RALFSIALES Nakamura, 1972 emend. Lin et Kawai (61), the family Lithodermataceae Hauck,1883, the genus Pseudolithodeima Svedelius in Engler et Prantl, 1911 [subextensum (Waem) S. Lund], the family Ralfsciaceae Fardlow, 1881, the genus Analipus Kjellman, 1889 [filiformis (Ruprecht) Papenfuss, gunjii (Yendo) Kogame et Yoshida, Chordaria gunjii, japonicus (Harvey) Wynne, Heterochordaria abietina], the genus Diplura Hollenberg, 1969 [simplex J. Tanaka et Chilhara], the genus Endoplura Hollenberg, 1969 [aurea Hollenberg], the genus Hapalospongidion Saunders, 1899 [schmidtii (Weber-van Bosse) Silva, Mesospora schmidtii], the genus Heteroralfsia Kawai, 1989 [saxicola (Okamura et Yamada) Kawai, Gobia saxicola, Saundersella saxicola], the genus Ralfsia Berkeley in Smith et Sowerby, 1843 [fungiformis (Gunnerus) Setchell et Gardner, verrucosa (Areschoug) Areschoug]; the order SYRINGODERMATALES Henry, 1984, the family Synringodermataceae Henry, 1084, the genus Syringoderma Levring, 1940 [abyssicola (Setchell et Gardner) Levring]; the order SPHACELARIALES Migula 1909, the family Sphacelariaceae Decaisne, 1842, the genus Sphacelaria Lyngbye in Hornemann, 1818 [californica Sauvageau, cornuta Sauvageau, divaricata Montagne, furcigera sensu Reinke, hizikiae, prostrata, radicans sensu Yendo, tenuis, nipponica Kitayama, plumigera Holmes ex Hauck, rigidula Kuetzing, apicalis, caespitosa, expansa, furcigera, iridaeophytica, iwagasakensis, linearis, sessilis, subfusca, variabilis, solitaria (Pringsheim) Kylin, divaricata, f. japonica, shiiyaensis, viridis, tribuloides Meneghini, yamadae Segawa, pyriformis, radiata], the family Stypocaulaceae Oltmanns, 1922, the genus Halopteris Kuetzing, 1843 [filicina (Grateloup) Kuetzing], the genus Stypocaulon Kuetzing, 1843 [durum (Ruprecht) Okamura]; the order DICTYOTALES Kjellman in Engler et Prantl, 1896, the family Dictyotaceae Lamouroux ex Dumortier, 1822, the genus Dictyopteris Lamouroux, 1809 nom. cons. [divaricata (Okamura) Okamura, fucoides Tanaka, latiuscula (Okamura) Okamura, papenfussii Tanaka, plagiogramma (Montagne) Vickers, polypodioides (De Candolle) Lamouroux, membranacea, prolifera (Okamura) Okamura, punctata Noda, repens (Okamura) Boergesen, undulata Holmes], the genus Dictyota Lamouroux, 1809 nom. cons. [bartayresii Lamouroux, dentata Lamouroux, dichotoma (Hudson) Lamouroux, dilatata Yamada, divaricata Lamouroux, friabilis Setchell, linearis (C. Agardh) Greville, maxima Zanardini, patens J. Agardh, spathulata Yamada, spinulosa Harvey], the genus Distromium Levring, 1940 [decumbens (Okamura) Levring, Chlanidophora repens, Chlanidote decumbens], the genus Homoeostrichus J. Agardh, 1894 [flabellatus Okamura], the genus Lobophora J. Agardh, 1894 [variegata (Lamouroux) Womersley ex Oliveira, Gymnosorus collaris, Pocockiella variegata], the genus Pachydictyon J. Agardh, 1984 [coriaceum (Holmes) Okamura], the genus Padina Adanson, 1763 nom. cons. [arborescens Holmes, australis Hauck, boryana Thivy, crassa Yamada, fasciata Ni-Ni-Win, Arai et Kawai, japonica Yamada, melemele Abbot et Magruder, minor Yamada, moffittiana Abbot et Huisman, okinwaensis Ni-Ni-Win, Arai et Kawai, ryukyuana Y. P. Lee et Kamura, stipitata Tanaka et Nozawa, terricolor Ni-Ni-Win, Arai et Kawai, undulata Ni-Ni-Win, Arai et Kawai], the genus Ruglopteryx DeClerk et Coppjans 2009 [okamurae (Dawson) Hwang, Lee et Kim in Hwang et al., Dilophus okamurae, Dilophus marginatus], the genus Spatoglossum Kuetzing, 1843 [crassum J. Tanaka, variabile sensu Yendo, latum J. Tanaka, pacificum Yendo, cornigerum sensu Yendo, solieri sensu Yendo], the genus Stypopodium Kuetzing, 1843 [zonale (Lamouroux) Papenfuss, lobatum], the genus Zonaria C. Agardh, 1817 nom. cons. [diesingiana J. Agardh, stipitata Tanaka et K. Nozawa]; the order CHORDARIALES Setchell et Gardner, 1925, the family Acrotrichaceae Kuckuck, 1929, the genus Acrothrix Kylin, 1907 [gracilis Kylin, pacifica Okamura et Yamada], the family Chordariaceae Greville, 1830, the genus Chordaria C. Agardh, 1817 nom. cons. [chordaeformis (Kjellman) Kawai et Kim, flagelliformis (O. F. Mueller) C. Agardh, gracilis Setchell et Gardner], the genus Cladosiphon Kuetzing, 1843 [Okamuranus Tokida, Eudesme virescens sensu Okamura, umezakii Ajisaka], the genus Eudesme J. Agardh, 1882 [virescens (Carmichael ex Berkeley) J. Agardh], the genus Heterosaundersella Tokida, 1942 [hattoriana Tokida], the genus Myriogloea Kuckuck ex Oltamanns, 1922 [simplex (Segawa et Ohta) Inagaki], the genus Papenfussiella Kylin, 1940 [kuromo (Yendo) Inagaki, Myriocladia kuromo], the genus Saundersella Kylin, 1940 [simplex (Saunders) Kylin, Gobia simplex], the genus Sauvageaugloia Hamel ex Kylin, 1940 [ikomae (Narita) Inagaki], the genus Sphaerotrichia Kylin, 1940 [divaricata (C. Agardh) Kylin, japonica, sadoensis, Castagnea divaricata, Chordaria cladosiphon, firma (E. Gepp) Zinova (20), Chordaria firma], the genus Tinocladia Kylin. 1940 [crassa (Suringar) Kylin, Eudesme crassa], the family Elachistaceae Kjellman, 1890, the genus Elachista Duby, 1830 nom. et orth. cons. [coccophorae Takamatsu, orbicularis, Gonodia orbicularis, Okamura Yoshida, fucicola sensu Okamura, globosa Takamatsu, taeniaeformis Yamada, crassa, flaccida auct. Japon., sadoensis, tenuis Yamada, vellosa Takamatsu], the genus Halothrix Reinke, 1888 [ambigua Yamada, lumbricalis sensu Takamatsu, tortuosa], the genus Leptonematella Silva, 1959 [fasciculata (Reinke) Silva], the family Leathesiaceae Farlow, 1881, the genus Kurogiella Kawai, 1993 [saxicola Kawai], the genus Leathesia S. F. Gray, 1821 [crassipilosa Takamatsu, difformis (Linnaeus) Areschoug, monilicellulata Takamatsu, primaria Takamatsu, pulvinata Takamatsu, sadoensis Inagaki, saxicola Takamatsu, granulosa], the genus Phaeophysema Tanaka, Uwai et Kawai 2010 [pulvinata (Takamatsu) Tanaka, Uwai et Kawai, sphaerocephala (Yamada) Tanaka, Uwai et Kawai], the genus Vimineolaethesia Tanaka, Uwai et Kawai[japonicaa (Inagaki) Tanaka, Uwai et Kawai], the genus Myriactula Kuntze, 1898 [sargassi (Yendo) Feldmann, saromaensis Yamada et Iwamoto], the family Petrospongiaceae Recault in Recault et al. 2009, the genus Petrospongium Naegeli ex Kuetzing, 1858 [rugosum (Okamura) Setchell et Gardner, Cylindrocarpus rugosus], the family Myrionemataceae Naegeli, 1847, the genus Compsonema Kuckuck, 1899 [nummuloides Setchell et Gardner], the genus Hecatonema Sauvageau [maculans (Collins) Sauvageau, terminate (Kuetzing) Kylin], the genus Microspongium Reinke, 1888 [Myrioncma globosum], the genus Myrionema Greville, 1827[corunnae Sauvageau], the genus Protectocarpus Kuckuck et Kornmann, 1955 [speciosus (Boergesen) Kuckuck ex Kornmann, Compsonema ramulosum sensu Noda], the family Spermatochnaceae Kjellman, 1890, the genus Nemacystus Derbes et Solier, 1850 [decipiens (Suringar) Kuckuck], the genus Stilophora J. Agardh, 1841 nom. cons. [tenella (Esper) Silva, rhizodes]; the order DICTYOSIPHONALES Setchell et Gardner, 1925, the family Asperococcaceae Farlow, 1881, the genus Asperococcus Lamouroux, 1813 [bullosus Lamouroux, turneri], the genus Melanosiphon Wynne, 1969 [intestinalis (Saunders) Wynne, Myelophycus intestinalis], the family Coilodesmaceae Setchell et Gardner, 1925, the genus Coilodesme Stroemfelt, 1886 [cystoseirae (Ruprecht) Setchell et Gardner, japonica Yamada, cystoseirae sensu Yendo], the family Delamareaceae A. D. Zinova, 1953, the genus Delamarea Hariot, 1889 [attenuata (Kjellman) Rosenvinge], the genus Stschapovia A. D. Zinova, 1954, the family Dictyosiphonaceae Kuetzving, 1849, the genus Dictyosiphon Greville,1830 nom. cons. [chordaria Areschoug, foeniculaceus (Hudson) Greville, hippuroides], the family Punctariaceae (Thuret) Kjellman, 1880, the genus Pogotrichum Reinke,1892 [yezoense (Yamada et Nakamura) Sakai et Saga, Litosiphon yesoense], the genus Punctaria Greville, 1830 [flaccida Nagai, kinoshitae Yamada et Iwamoto, tenuis, latifolia Greville, mageshimensis Tanaka, occidentalis Setehell et Gardner, chartacea sensu Umezaki, conglomerata, pilosa Umezaki, plantaginea (Roth) Greville, rubescens sensu Yendo, projecta Yamada], the genus Trachynema Pedersen, 1985 [groenlandicum (Lund) Pedersen], the family Striariaceae Kjellman, 1890, the genus Coelocladia Rosenvinge, 1893 [arctica Rosenvinge], the genus Stictyosiphon Kuetzing, 1843 [soriferus (Reinke) Rosenvinge, Kjellmania arasakii], the genus Striaria Greville, 1828 [attenuata (Greville) Greville]; the order SCYTOSIPHONALES J. Feldmann, 1949, the family Chnoosporaceae Setchell et Gardner, 1925, the genus Chnoospora J. Agardh, 1847 [implexa J. Agardh, minima (Hering) Papenfuss, pacifica], the family Scytosiphonaceae Farlow, 1881, the genus Colpomenia (Endlicher) Derbes et Solier in Castagne, 1851 [bullosa (Saunders) Yamada, sinuosa f. deformans, peregrina (Sauvageau) Hamel, phaeodactyla Wynne et J. N. Norris, sinuosa (Mertens ex Roth) Derbes et Solier], the genus Hydroclathrus Bory, 1825 [clathratus (C. Agardh) Howe, tenuis Tseng et Lu], the genus Myelophycus Kjellman in Engler et Prantl, 1893 [cavus J. Tanaka et Chihara, simplex (Harvey) Papenfuss, caespitosus], the genus Petalonia Derbes et Solier, 1850 nom. cons. [binghamiae (J. Agardh) Vinogradova, Endarachne binghamiae, fascia (O. F. Mueller) Kuntze, Ilea fascia, zosterifolia (Reinke) Kuntze], the genus Rosenvingea Boergesen, 1914 [intricata (J. Agardh) Boergesen], the genus Scytosiphon C. Agardh, 1820 nom. cons. [canaliculatus (Setchell et Gardner) Kogame, gracilis Kogame, lomentaria (Lyngbye) Link, tenellus Kogame]; the order CUTLERIALES Oltmanns, 1922, the family Cutleriaceae Hauck, 1883, the genus Cutleria Greville, 1830 [adspersa (Roth) De Notaris, cylindrica Okamura, multifida (Turner) Greville]; the order SPOROCHNALES Sauvageau, 1926, the family Sporochanaceae Greville, 1830, the genus Carpomitra Kuetzing, 1843 nom. cons. [costata (Stackhouse) Batters, cabrerae], the genus Nereia Zanardini, 1846 [intricata Yamada], the genus Sporochnus C. Agardh, 1817 [radiciformis (R. Brown ex Turner) C. Agardh, scoparius]; the order DESMARESTIALES Setchell et Gardner, 1925, the family Desmarestiaceae (Thuret) Kjellman, 1880, the genus Desmarestia Lamouroux, 1813 nom. cons. [ligulata (Stackhouse) Lamouroux, tabacoides Okamura, viridis (Mueller) Lamouroux]; the order LAMINARIALES Kylin, 1917, the family Akkesiphycaceac Kawai et Sasaki, 2000, the genus Akkesiphycus Yamada et Tanaka, 1944 [lubricus Yamada et Tanaka], the family Alariaceae Setchell et Gardner, 1925, the genus Alaria Greville, 1830 nom. cons. [angusta Kjellman, crassifolia Kjellman, paradisea (Miyabe et Nagai) Widdowson, Pleuropterum paradiseum, praelonga Kjellman, taeniata Kjellman], the genus Druehlia Lane et Saunders, 2007 [fistulosa (Postels et Ruprecht) Lane et Saunders], the genus Undaria Suringar, 1873 [peterseniana (Kjellman) Okamura, pinnatifida (Harvey) Suringar, undarioides (Yendo) Okamura], the family Chordaceae Dumortier, 1822, the genus Chorda Stackhouse, 1797 [asiatica Sasaki et Kawai, Chora filum auct. japon.], the genus Agarum Dumortier, 1822 nom. cons. [clathratum Dumortier, cribrosum, oharaense Yamada], the genus Costaria Greville, 1830 [costata (C. Agardh) Saunders], the family Laminariaceae Bory, 1827, the genus Ecklonia Hornemann, 1828 [cava Kjellman, kurome Okamura, stolonifera Okamura], the genus Eckloniopsis Okamura, 1927 [radicosa (Kjellman) Okamura], the genus Eisenia Areschoug, 1876 [arborea Areschoug, bicyclis (Kjellman) Setchell], the genus Arthrothamnus Ruprecht, 1848 [bifidus (Gmelin) Ruprecht], the genus Cymathaere [Cymathaere japonica, crassifolia sensu Miyabe et Nagai], the genus Laminaria Lamouroux, 1813 nom. cons. [yezoensis Miyame], the genus Saccharina Stackhouse 1809 [angustata (Kjellman) Lane, Mayes, Druehl et Saunders, Laminaria angustata, cichorioides (Miyabe) Lane, Mayes, Druehl et Saunders, Laminaria cichorioides, coriacea (Miyabe) Lane, Mayes, Druehl et Saunders, Laminaria coriacea, diabolica (Miyabe) Lane, Mayes, Druehl et Saunders, Laminaria diabolica, gyrata (Kjellman) Lane, Mayes, Druehl et Saunders, Laminaria gyrata, Kjellmaniella gyrata, japonica (Areschoug) Lane, Mayes, Druehl et Saunders, Laminaria japonica, kurilensis Lane, Mayes, Druehl et Saunders, Cymathaere japonica Miyabe et Nagai, latissima (Linneaus) Lane, Mayes, Druehl et Saunders, Laminaria saccharina, longipedalis (Okamura) Lane, Mayes, Druehl et Saunders, Laminaria longipepedalis, longissima (Miyabe) Lane, Mayes, Druehl et Saunders, Laminaria longissima, ochotensis (Miyabe) Lane, Mayes, Druehl et Saunders, Laminaria ochotensis, religiosa (Miyabe) Lane, Mayes, Druehl et Saunders, Laminaria religiosa, Sculpera Lane, Mayes, Druehl et Saunders, Kjellmaniella crassifolia, Saccharina crassifolia, yendoana (Miyabe) Lane, Mayes, Druehl et Saunders, Laminaria yendoana], the genus Streptophyllopsis Kajimura, 1981 [kuroshioensis (Segawa) Kajimura, Hedophyllum kuroshioense], the family Pseudochordaceae Kawai et Kurogi, 1985, the genus Pseudochorda Yamada, Tokida et Inagaki in Inagaki, 1958 [gracilis Kawai et Nabata, nagaii (Tokida) Inagaki, Chordaria nagaii]; the order ISHIGEALES Cho et Boo in Cho et al., 2004, the family Ishigeaceae Okamura in Segawa, 1935, the genus Ishige Yendo, 1907 [okamurae Yendo, sinicola (Setchell et Gardner) Chihara, foliacea]; the order FUCALES Kylin, 1917, the family Fucaceae Adanson, 1763, the genus Fucus Linnaeus, 1753 [distichus Linnaeus subsp. evanescens (C. Agardh) Powell, evanescens], the genus Silvetia Serrao, Cho, Boo et Brawley in Serrao et al. 1999 [babingtonii (Harvey) Serrao et al., Pelvetia babingtonii, Pelvetia wrightii], the family Sargassaceae Kuetzing, 1843, the genus Coccophora Greville, 1830 [langsdorfii (Turner) Greville], the genus Cystoseira C. Agardh, 1820 nom. cons. [crassipes (Mertens ex Turner) C. Agardh, Cystophyllum crassipes, geminata C. Agardh, Cystophyllum geminatum, hakodatensis (Yendo) Fensholt, Cystophyllum hakodatense], the genus Hormophysa Kuetzing, 1843[cuneiformis (Gmelin) Silva, triquetra, Cystoseira proliferal], the genus Myagropsis Kuetzing, 1843 [myagroides (Mertens ex Turner) Fensholt, yendoi, Cystophyllum caespitosum, Cystophyllum sisymbrioides, Cystophyllum turneri], the genus Sargassum C. Agardh, 1820 nom. cons. [alternato-pinnatum Yamada, asymmetricum, ammophilum Yoshida et I. Konno, araii Yoshida, assimile Harvey, autumnale Yoshida, boreale Yoshida et Horiguchi, bulbiferum Yoshida, carpophyllum J. Agardh, angustifolium sensu Yamada, vulgare var. linearifolium sensu Yendo, confusum C. Agardh, crassifolium J. Agardh, berberifolium, crispifolium Yamada, cristaefolium C. Agardh, denticarpum Ajisaka, duplicatum Bory, brevfolium sensu Yendo, sandei, filicinum Harvey, fulvellum (Turner) C. Agardh, enerve, fusiforme (Harvey) Setchell, Hizikia fusiformis, giganteifolium Yamada, glaucescens J. Agardh, hemiphyllum (Turner) C. Agardh, hornei (Turner) C. Agardh, ilicifolium (Turner) C. Agardh var. conduplicatum Grunow, incanum Grunow, kashiwajimanum Yendo, kushimotense Yendo, longifructum Tseng et Lu, macrocarpum C. Agardh, serratifolium auct. japon., micracanthum (Kuetzing) Endlicher, microceratium (Mertens ex Turner) C. Agardh, miyabei Yendo, kjellmanianum, muticum (Yendo) Fensholt, kjellmanianum f. muticum, myriocystum J. Agardh, opacum, nigrifolium Yendo, nipponicum Yendo, okamurae Yoshida et T. Konno, pallidum (Turner) C. Agardh, patens C. Agardh, piluliferum (Turner) C. Agardh, pinnatifidum Harvey, polycystum C. Agardh, polyporum Monagne, ssp. ringgoldianum, ssp. coreanum (J. Adardh) Yoshida, ryukyuense Shimabukuro et Yoshida, sagamianum Yendo, salicifolioides Yamada, hyugaense, segii Yoshida, racemosum Yamada et Segi, ringgoldianum f. ellipticum, serratifolium (C. Agardh) C. Agardh, siliquastrum (Turner) C. Agardh, tortile, siliquosum J. Agardh, spathulophyllum J. Tanaka, Murakami et Arai, tenuifolium Yamada, thunbergii (Mertens ex Roth) Kuntze, tosaense Yendo, trichophyllum (Kuetzing) Kuntze, wakayamaense Yoshida, yamadae Yoshida et T. Konno, Yamamotoi Yoshida, yendoi Okamura et Yamada, henslowianum var. condensatum, yezoense (Yamada) Yoshida et T. Konno, sagamianum var. yezoense], and the genus Turbinaria Lamouroux, 1825 [conoides (J. Agardh) Kuetzing, ornata (Turner) J. Agardh, turibinata (Linnaeus) Kuntze, trialata]. In the methods of the present invention, hormeri (Turner) C. Agardh, cava Kjellman, kurome Okamura, and kelp (Laminariaceae Bory) are preferred, with hormeri (Turner) C Agardh being the most preferred.

The brown algae to be used as the starting material in the methods of the present invntion are cut to a size of about 1 m without performing a drying treatment after harvest and then washed with water as such, followed by draining for immediate use; alternatively, the brown algae as cut to a size of about 1 m are stored frozen as such at −20° C. or below, thawed just before use, and then put to use. The brown algae either as harvested or as simply stored frozen are hereinafter referred to as the untreated brown algal starting material. In order to reduce its iodine content, a dried product of the stating material has to be first reconstituted with water before it is deprived of iodine by treatment with water either at ordinary temperature or as heated but then the proccess becomes complicated. What is more, the effectiveness of this technique for removing iodine is variable with the kind of seaweed and consistent reduction of the iodine content cannot be achieved, with the result that the iodine content is reduced only insufficiently; as a further problem, pigment components may potentially be decomposed during the process of drying and this is by no means preferable (see Non-Patent Document 6).

The present invention requires a treatment for shredding the untreated brown algal starting material. If the starting material is in a frozen state, it is thawed under flowing water and then shredded. Shredding may be performed by cutting or otherwise dividing the starting material to a size of about 1-3 cm as it is placed on a 9.6 mm strainer. The shredded brown alga is then added into hot water at 90° C. or above, followed by stirring to effect treatment with the hot water. This combination of shredding and the treatment with hot water enables efficient reduction of the iodine content. If shredding is performed without treatment with hot water, or if treatment with hot water is performed without shredding, the intended effects of the present invention will not be obtained. The time of treatment with hot water is so chosen as to provide the desired percent residue of pigment components and the desired percent residue of iodine. To be more specific, the amounts of pigment components are so chosen as to be beneficiary for intake by humans whereas the amount of iodine is so chosen as to prevent excessive intake. For example, the effective intake of the pigment component chlorophyll c is specified to be 0.1-100 mg/day, desirably 0.7-0.9 mg/day, on a dry weight basis (Paragraph No. [0036] of Patent Document 1) and the reference intake of iodine is specified to be no more than 2.2 mg/day (adult) (see Dietary Reference Intakes for Japanese 2010, Ministry of Health, Labour and Welfare), so the effective amount of chlorophyll c can be ingested without causing damage to health if chlorophyll c/iodine is between 0.32 to 0.41 and above at the point in time when the heating treatment ends. It should be noted here that the reference intake of iodine varies from country to country and in U.S., it is prescribed to be 1.1 mg/day (Dietary Reference Intakes (2001), The National Akademies) and in EU, it is prescribed to be 0.6 mg/day (National Institute of Health Sciences, Divisiojn of Safety Information on Drug and Food, Safety Information on Food (Chemicals) No. 5/2013 (Mar. 6, 2013) P10). As for the pigment component fucoxanthin, its effective intake is specified to be 0.5-1.0 mg/day (JP 2008-291004A) and the effective amount of fucoxanthin can be ingested without causing damage to health if the fucoxanthin/iodine ratio is between 0.23 to 0.45 and above. The treatment time for achieving the above-noted reference intakes is typically 0.5-5 minutes, more preferably 1.5-5 minutes. If the treatment time is less than 0.5 minutes, the iodine content cannot be sufficiently reduced. If, on the other hand, the treatment time exceeds 5 minutes, the pigment components are so much decomposed that the intended effects of the present invention cannot be attained. The temperature of hot water is preferably between 90° C. and 100° C. Temperatures less than 90° C. are not preferred since iodine does not dissolve out sufficiently. Hot water is desirably used in amounts at least five times that of the brown alga to be processed. If the amount of hot water is less than five times that of the brown alga, it again occurs that the iodine content is reduced only insufficiently.

Thus, it has been found that pigment components in brown algae are not decomposed even if they are shredded and added into hot water at 90° C. and above, followed by stirring to effect treatment with the hot water. In this connection, it is interesting to note that the pigment component chlorophyll c present in brown algae may be compared in structure to chlorophylls a, b and d, which have varying degrees of heat stability depending on structure. The structures of these chlorophylls are depicted below.

Formula 1
Molecular structures of chlorophylls
a)

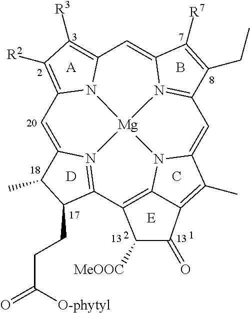

| Pigment | R² | R³ | R⁷ |
|---|---|---|---|
| Chl a | Me | CH₂═CH | Me |
| Chl b | Me | CH₂═CH | CHO |
| Chl d | Me | CHO | Me |
| Chl f | CHO | CH₂═CH | Me |

b)

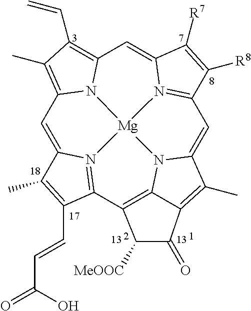

| Pigment | R⁷ | R⁸ |
|---|---|---|
| Chl c₁ | Me | Et |
| Chl c₂ | Me | CH₂═CH |
| Chl c₃ | COOMe | CH₂═CH |

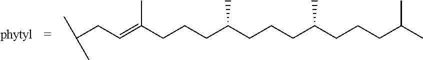 [SMILES: C/C(=C\CC(C)C)CCC[C@H](C)CCC[C@H](C)CCCC(C)C]

As regards heat stability, it is known that chlorophylls b and d which have a CHO— group in the porphyrin skeleton are stable whereas chlorophyll a which has no CHO— group in the porphyrin skeleton is labible (Chem. Biodiver. Vol. 9, 1659-1683, 2012). Hence, chlorophyll c which, like chlorophyll a, has no CHO— group in the porphyrin skeleton is predicted to be heat labible. Yet, surprisingly enough, even when brown algae were treated with hot water, their chlorophyll c was found to be equally stable or more stable than the highly heat stable chlorophyll b which occurs in green vegetables. To be more specific, the percent chlorophyll residue after 5-min heating at 100° C. was reported to be about 90% for garland chrysanthemum, about 92% for podded peas, about 85% for spinach, and about 70% for green peas (Chori Kagaku (Cookery Science), Vol. 9, No. 2, pp. 53-58 (1976)) whereas the brown alga hormeri (Turner) C. Agardh as treated in hot water at 90° C. and above had at least 95% chlorophyll residue.

As described above, the iodine content in the untreated brown algae can be efficiently reduced by combining shredding and the treatment with hot water. If either one of the treatments is omitted, the iodine content is reduced only insufficiently.

The first step is described below more specifically.

The untreated brown alga (with water content of 90 wt %) that has been stroed as described above is thawed under flowing water and thereafter shredded to a size of about 1-3 cm (on 9.6 mm strainer). The shredded brown alga is added into hot water at 90-100° C., which is gently stirred for 90-300 seconds. Thereafter, the treated brown alga is recovered and immersed in cold water at 10° C. or below, where it is cooled and washed. After washing with water, the brown alga is recovered and subjected to centrifugal dehydration to dry. Depending on the need, the resulting dried brown alga is ground to a desired size, say, about 200 mesh and finer, whereupon a brown algal powder is obtained.

The brown algal powder obtained by the above-described produciton method has the iodine content reduced by 90% or more as compared with the untreated seaweed and yet the pigment components remain barely decomposed.

The present invention also lies in a method for producing a brown algal extract of reduced iodine content while retaining pigment components in the brown alga, which method is characterized in that the heat treated brown alga as obtained in the manner described above is subjected to extraction using ethanol at a concentration of 60-100%.

The extraction step described above aims to increase the contents of pigment components while further reducing the iodine content. The concentration of ethanol is between 60% and 100%, preferably between 80% and 100%, and most prferably between 90% and 100%. If the ethanol concentration is less than 60%, not only is the percent transfer of the pigment components into the liquid extractant insufficient but also the percent transfer of iodine is high and the object of the present invention cannot be attained. As a further problem, sticky components are extracted in so large amounts that process operability is impaired. The extraction time and temperature are of values that are sufficient to prevent the extraction of iodine while assuring adequate extraction of the pigment components; for example, the extracton time is 1-16 hours, preferably 1-2 hours, and most preferably 2 hours, whereas the extraction temperaure is between ordinary temprature and 80° C., preferably about 70° C.

Iodine (I₂) is known to be easily soluble in ethanol (24 g/100 g, 25° C.) but difficult to dissolve in water (0.029 g/100 mL, 20° C.) (Safety Data Sheet, Showa Chemical Co., Ltd., Reagent's Information). It has been reported that the iodine contained in brown algae such as hormeri (Turner) C. Agardh exists outside cells (apoplast) as the molecular I₂ or HOI which is chelated to sulfated polysaccharides and so on (Abstracts of Lectures read at the 2012 Autumn Symposium on Algae, in Kaiso Shigen (Seaweed Resources), No. 27, “The effects on health of iodine and arsenic contained in seaweeds”.) It has also been reported that kelp (Laminariaceae Bory) contains a volatile iodine compound (see the formula below) (Nihon Shokuhin Kagaku Kogaku Kaishi (Journal of the Japanese Society for Food Science and Technology), Vol. 49, No. 4, 2002.)

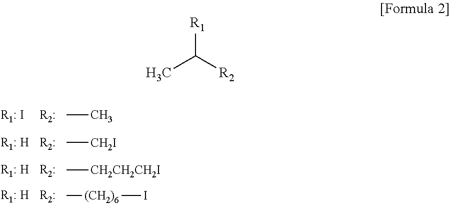

Volatile Iodine Compound

The volatile iodine compound, like I₂, is also difficult to dissolve in water and easily soluble in ethanol, so whichever form of iodine that may be contained in brown algae would show similar behavior with respect to water and ethanol (Safety Data Sheet (JUNSEI CHEMICAL CO., LTD.) and Reagent's Information (Merck Japan)). It is, therefore, predictable from these sets of known information that iodine is easily soluble in ethanol and can readily be extracted with ethanol. Yet, surprisingly enough, it has been found in the present invention that iodine is difficult to dissolve in ethanol at concentrations of 60-100%.

Althouogh not wishing to be bound by theory, the present inventors assume that since it occurs in brown algae as chelated to water-soluble polysaccharides, iodine is difficult to dissolve in high concentrations of ethanol on account of the interference by the polysaccharides.

The second step is described below more specifically.

The brown algal extract obtained in the first step is added to a liquid extractant having an ethanol concentration of 60-100% at a specified temperature (say, 70° C.), which is stirred over a specified time (say, 1-2 hours) to effect extraction. After the extraction, the liquid extractant is filtered to remove any residue, recovered and dried under reduced pressure to yield a brown algal extract. Depending on the need, the recovered liquid extractant may be mixed with an excipient and then dried under reduced pressure to give a powder form.

The extract powder recovered in the second step as described above contains 50 mg or more of chlorophyll c per 100 g and in a more preferred embodiment, it contains 100 mg or more of the pigment components. On the other hand, the iodine content can be reduced to 100 mg and less per 100 g, whereby the extract powder obtained has a chlorophyll c/iodine ratio of 0.32 and more. It should be notd that chlorophyll c and fucoxanthin can be quantitatively measured by HPLC whereas iodine can be quantitatively measured by gas chromatography, etc.

Thus, in accordance with the extraction method of the present invention, the pigment components can be extracted efficiently without extracting the excess iodine that remains in the brown alga or brown algal powder that have passed through the first step.

The present invention also relates to brown algae or brown algal extracts that have been obtained by the methods described above. These brown algae or brown algal extracts, either as such or after addition of suitable additives, may be adapted into various forms for use as foods, beverages, and so on. The applicable form is not particularly limited. Examples include dusts, granules, tablets, syrups, powders, suspensions, etc. The above-mentioned formulations may contain additives such as excipients, disintegrants, binders, lubricants, and colorants. Exemplary excipients include lactose, glucose, corn starch, sorbitol, and microcrystalline cellulose; exemplary disintegrants include starch, sodium alginate, gelatin powder, calcium carbonate, calcium citrate, and dextrin; exemplary binders include dimethyl cellulose, poly(vinyl alcohol), poly(vinyl ether), methylcellulose, ethyl cellulose, gum arabic, gelatin, hydroxypropyl cellulose, and poly(vinyl pyrrolidone); exemplary lubricants include talc, magnesium stearate, poly(ethylene glycol), and hardened vegetable oils.

Depending on their dosage form, the formulations of the present invention usually contain pigment components such as chlorophyll c and fucoxanthin in amounts of about 0.1 mg to 100 mg by dry weight in the total formulation.

The amount of intake is determined as appropriate for individual cases in consideration of various factors such as the age, body weight, sex, and physical condition of the user, and the frequency of intake may be once or more than once a day. For instance, chlorophyll c may be ingested in a dry weight ranging from 0.1 mg to 100 mg, and an algal powder or an algal extract that contains chlorophyll c as an active ingredient may be ingested in a dry weight ranging from about 0.1 g to 10 g, preferably from about 1 g to 3 g. In the case of fucoxanthin, the amount of its use can be varied depending on the method of use, a user's physical condition and age, etc.; for adults, fucoxanthin as active ingredient may typically be used in daily amounts from about 5 mg to about 200 mg, and for children, in daily amounts from about 0.5 mg to about 100 mg (JP 2008-291004A).

The brown algae or brown algal extracts of the present invention may, either on its own or after being added to processed foods, serve as functional foods or beverages that have degranulation suppressing activity, allergy suppressing activity or osteoarthritis suppressing activity, and the type of the foods or beverages to be produced is not particularly limited if they do not interfere with the degranulation suppressing action, allergy suppressing action or osteoarthritis suppressing action of the active ingredient chlorophyll c. In the case of fucoxanthin, it may be incorporated in foods or beverages, cosmetics, animal feeds, and so on (JP 2008-291004A, JP 2010-275265A, and JP 2012-224602A).

Examples of the processed food to which the brown algae or brown algal extracts of the present invention may be added include: bakery; cereal flour and noodles; processed fishery products; processed agricultural and forestry products; processed animal foods; cow's milk and dairy products; oils and fats, either unprocessed or processed; liquors; beverages; seasonings; prepared frozen foods; retorted foods; instant foods; seasoned dried products such as fish/shell senbei; salt preserved fish roe and other products; tsukudani (fish and other foods boiled in soy sauce) such as kanroni, shigureni, and kakuni; broiled processed products; boiled processed products; seasoned processed products such as fish/shell miso; kamaboko (minced and steamed fish) such as mushi(steamed)-kamaboko, yaki(broiled)-kamoboko, age (fried)-kamaboko, yude(boiled)-kamaboko, fuumi(flavored)-kamaboko, housou(packaged)-kamaboko, saiku(crafted)-kamaboko, and kunsei(smoked)-kamaboko; pickles such as shoyu(soy)-zuke, miso-zuke, kasu(sake lees)-zuke, su(vinegar)-zuke, and koji(rice malt)-zuke; shiokara (salted fish guts) such as katsuo(bonito)-shiokara, uni(sea urchin)-shiokara, and ika(squid)-shiokara; canned products; binned products; fish sauce; and extract products.

The food and beverage of the present invention encompass health foods and beverages, dietary supplements, foods for specified health uses, foods with nutrient functional claims, etc. The “foods for specified health uses” refers to those foods and beverages that are ingested in dietary life for a specified health purpose and which have a label claiming that the specified health purpose may be achieved by ingesting them. Such foods and beverages may have labels attached thereto, claiming that they “alleviate symptoms of allergy,” “alleviate symptoms of pollinosis,” “alleviate symptoms of atopic dermatitis,” “alleviate symptoms of osteoarthritis,” or that they are “foods for persons worried about allergy” or “foods for persons worried about osteoarthritis.” In the case of fucoxanthin, labels may be attached that claim, for example, “Best for persons with metabolic syndrome,” “For in vivo antioxidation,” “Ingredient of beauty food,” or “Ingredient of cosmetic.”

The brown alga or brown algal extract of the present invention may, either on their own or after being added to a feed or pet food, serve as a feed or pet food that has degranulation suppressing activity, allergy suppressing activity or osteoarthritis suppressing activity. The type of the feed or pet food to be produced is not particularly limited if it does not interfere with the degranulation suppressing action, allergy suppressing action or osteoarthritis suppressing action of the active ingredient chlorophyll c.

The feed or pet food of the present invention is not particularly limited in scope and they may be feeds for cattle and poultry such as cows, pigs and chickens, or feeds for farming crustaceans and fish/shellfish, or pet foods for companion animals such as dogs, cats, hamsters, and squirrels. The form the feed or pet food of the present invention can take is not particularly limited and examples include a pellet type, a crumble type, a flake type, a bulky type, a dry type, a wet type, a semi-moist type, a biscuit type, a sausage type, a jerky type, a powder type, a granule type, a capsule type, etc.

### EXAMPLES

The present invention will be described below more specifically by reference to Examples. It should, however, be understood that the following Examples are by no means intended to limit the scope of the present invention.

(Measurement of the Chlorophyll c and Fucoxanthin Contents)

Based on calibration curves, the chlorophyll c and/or fucoxanthin content in the above-described extract was quantified under the following HPLC conditions to calculate the chlorophyll c and fucoxanthin in a processed seaweed, a processed seaweed powder, and an extract powder.

<HPLC Conditions>

Pump: 2695 Separations Module (Nihon Waters K.K.)

Detector: 2998 Photodiode Array Detector (Nihon Waters K.K.)

Column: XBridge BEH300 C18 Column, 3.5 μm, 4.6 mm×150 mm (Nihon Waters K.K.)

Mobile phase: 90% acetonitrile/80% methanol in 0.1M ammonium acetate/ethyl acetate

Flow rate: 1 mL/min

Column temperature: 30° C.

Detection wavelength: 450 nm

(Quantification of Iodine)

Quantification of iodine was carried out by gas chromatography (6890N (Agilent Technologies Company)). The detection limit was 0.5 ppm.

### Example 1

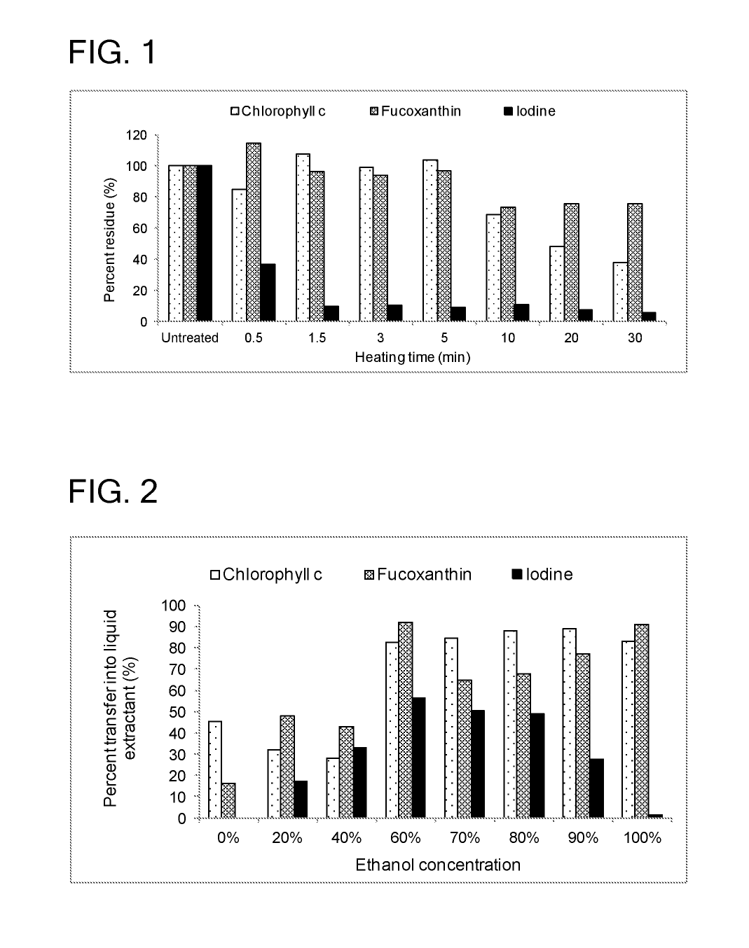

(Substep for Treatment with Hot Water in the First Step) Harvested hormeri (Turner) C. Agardh was frozen as such at −20° C. or below; 100 g of the frozen untreated hormeri (Turner) C. Agardh (with a water content of 90 wt %) was thawed under flowing water and thereafter shredded to sizes of about 1-3 cm on a strainer having an opening size of 9.6 mm. The shredded hormeri (Turner) C. Agardh was subjected to a heating treatment with 1000 mL of stirred hot water at 90° C. or higher over periods of 0.5 min, 1.5 min, 3 min, 5 min, 10 min, 20 min, and 30 min. The resulting hot water treated hormeri (Turner) C. Agardh was cooled to room temperature, then immersed in 1000 mL of fresh water at 10° C. or lower where it was cooled and washed for 5 minutes; thereafter, hormeri (Turner) C. Agardh was recovered, dehydrated with a centrifugal separator for 9 minutes, and dried at 70° C. for 6 hours. Subsequen grinding to about 200 mesh and finer gave a processed hormeri (Turner) C. Agardh powder. This processed hormeri (Turner) C. Agardh powder was measured for fucoxanthin and chlorophyll c and their percent residues are shown in FIG. 1, with the numerical value for untreated hormeri (Turner) C. Agardh being taken as 100. The chlorophyll c/iodine ratios for the respective test groups are also shown in Table 1.

TABLE 1

| | Iodine | Chlorophyll | |
|---|---|---|---|
| | (mg) (/100 | c (mg) (/100 | Chlorophyll |
| Test group | g solids) | g solids) | c/Iodine |
| Untreated | 351.6 | 44.1 | 0.15 |
| Treated with hot water, 0.5 min | 128.6 | 35.5 | 0.28 |
| Treated with hot water, 1.5 min | 33.8 | 47.5 | 1.41 |
| Treated with hot water, 3 min | 36.0 | 43.7 | 1.22 |
| Treated with hot water, 5 min | 32.4 | 45.6 | 1.41 |
| Treated with hot water, 10 min | 38.6 | 30.1 | 0.78 |
| Treated with hot water, 20 min | 25.6 | 21.3 | 0.83 |
| Treated with hot water, 30 min | 19.3 | 16.7 | 0.86 |

### Results

As can be seen from FIG. 1, the percent residue of chlorophyll c in the processed seaweed that was treated with hot water for 0.5-5 minutes in Example 1 was 80% and higher as compared with the value of 100 for the untreated seaweed. However, upon treatment for 10 minutes and longer, the percent residue of chlorophyll c began to decrease, becoming less than 40% upon treatment with hot water for 30 minutes. A similar tendency was observed for fucoxanthin, so an optimum treatment time for maintaining the pigment components was found to be within 5 minutes.

On the other hand, upon treatment with hot water for 0.5 minutes, the percent residue of iodine dropped to less than 40% as compared with the value of 100 for the untreated seaweed, and upon treatment for 1.5 minutes and longer, the value decreased considerably to lower levels in the neighborhood of 10%.

From the foregoing, it became apparent with the substep for treatment with hot water in the first step that the treatment with hot water for 1.5 to 5 minutes achieved a considerable reduction in the iodine content while maintaining the contents of the pigment components.

Further, the chlorophyll c/iodine ratio was calculated for the treatment with hot water for 1.5 to 5 minutes on the basis of the value of 0.32 calculated from 0.7 mg/day (the effective intake of chlorophyll c) and 2.2 mg/day (the reference intake of iodine); it also became apparent that the numerical value of interest was 1.2 and more.

Hence, by using the method of the present invention (the first step), there can be conveniently produced brown algae that are reduced in iodine content and which contain the functional pigment components.

### Example 2

(Substep for Extraction Treatment in the Second Step)

In the substep for extraction treatment, 10 g of the processed hormeri (Turner) C. Agardh powder as obtained in the first step was added to 50 mL each of 0-100% hydrous ethanol solutions and extraction treatment was conducted at an elevated temperature of 70° C. for 2 hours. After the extraction, filtration was performed to remove the residue, which was recovered from the filter paper and meassured for the contents of chlorophyll c, fucoxanthin, and iodine in the residue. The residue was cooled and washed with cold water at 10° C. and below. Subsequently, the extract of the processed hormeri (Turner) C. Agardh was dehydrated and measured for chlorophyll c, fucoxanthin, and iodine. The percentages of chlorophyll c, fucoxanthin and iodine that transferred into the liquid extractant are shown in FIG. 2, with the respective values for those in the extract of the processed hormeri (Turner) C. Agardh powder being taken as 100. The chlorophyll c/iodine ratios for the respective test groups are also shown in Table 2.

TABLE 2

| Extractant | Quantities of components extracted from | | |
|---|---|---|---|
| EtOH con- | the starting material (mg/100 g) | | Chlorophyll |
| centration | Chlorophyll c | Iodine | c/Iodine |
| 100% | 38.1 | 0.5 | 82.3 |
| 95% | 37.8 | 3.3 | 11.4 |
| 90% | 40.9 | 10.7 | 3.8 |
| 80% | 40.4 | 19.0 | 2.1 |
| 70% | 38.8 | 19.6 | 2.0 |
| 60% | 37.9 | 21.9 | 1.7 |
| 40% | 12.7 | 12.8 | 1.0 |
| 20% | 14.7 | 6.7 | 2.2 |
| 0% | 20.8 | 0.0 | 0.0 |

### Result

As can be seen from FIG. 2, at ethanol concentrations of 60-100%, the percent transfer of chlorophyll c into the liquid extractant in Example 2 was more than 80% as compared with the value of 100 for the processed seaweed powder. However, at ethanol concentrations of 40% and below, the percent transfer of chlorophyll c decreased to less than 40%, indicating a substantial drop in the extraction efficiency. It similarly turned out that fucoxanthin could also be recovered efficiently by extraction at ethanol concentrations of 60-100%.

The percent transfer of iodine into the liquid extract, as compared with the value of 100 for the processed seaweed powder, was 27.5% at an ethanol concentration of 90% and 1.2% at an ethanol concentration of 100%. In contrast, at ethanol concentrations of 60-80%, the percent transfer of iodine was 50%, indicating the extraction of iodine together with the pigment components. Even when lower concentrations of ethanol were used, the chlorophyll c/iodine ratio was 0.32 and above but this approach turned out to be unsuitable for the method of the present invention since concomitant extraction of polysaccharides as sticky components considerably lowered the process operability.

From the foregoing, it became apparent with the substep for extraction treatment in the second step that use of 60-100% ethanol suppressed the extraction of iodine with ethanol and yet enabled the pigment components to be extracted efficiently.

Hence, by using the present invention, there can be conveniently produced brown algal extract powders that are reduced in iodine content and whch contain the functional pigment components at high concentrations.

1. A method for producing a brown alga of reduced iodine content while retaining a pigment component in the brown alga, which method comprises:
shredding an untreated brown algal starting material and thereafter performing a heating treatment with hot water at 90-100° C. for 90-300 seconds; and
subjecting the resulting heat treated brown alga to extraction using hydrous ethanol at a concentration of 60-100%.

2. (canceled)

3. The method according to claim 1, wherein the pigment component is chlorophyll c and/or fucoxanthin.

4. The method according to claim 1, wherein the chlorophyll c/iodine ratio is 0.32 or above, and the fucoxanthin/iodine ratio is 0.23 or above.

5. The method according to claim 1, wherein the brown alga is selected from the group consisting of hormeri (Turner) C. Agardh, cava Kjellman, kurome Okamura, and kelp (Laminariaceae Bory).

6.-7. (canceled)
